(19) United States
(12) Patent Application Publication
(10) Pub. No.: US 2022/0183576 A1
(43) Pub. Date: Jun. 16, 2022

(54) MEDICAL SYSTEM, INFORMATION PROCESSING DEVICE, AND INFORMATION PROCESSING METHOD

(71) Applicant: SONY GROUP CORPORATION, TOKYO (JP)

(72) Inventors: KAZUKI IKESHITA, TOKYO (JP); TAKAMI MIZUKURA, TOKYO (JP)

(21) Appl. No.: 17/441,435

(22) PCT Filed: Mar. 16, 2020
(86) PCT No.: PCT/JP2020/011463

§ 371 Date: Sep. 21, 2021

(30) Foreign Application Priority Data
Mar. 29, 2019 (JP) 2019-069136

(51) Int. Cl. A61B 5/026; A61B 34/10; A61B 1/045; G01N 21/17; G02B 21/06; G02B 21/36; G02B 23/24

(52) U.S. Cl. CPC A61B 5/0261; A61B 34/10; A61B 1/045; G02B 23/24; G02B 21/06; G02B 21/36; G01N 21/17

(57) ABSTRACT

A medical system (1) acquires a speckle image from an imaging means that images reflected light of coherent light from a subject. Furthermore, a first parameter value and a second parameter value different from each other are stored as a parameter for calculating a speckle index value that is a statistical index value for a luminance value of a speckle. Furthermore, in a case where a first mode is selected, the speckle index value is calculated on the basis of the speckle image and the first parameter value, and in a case where a second mode is selected, the speckle index value is calculated on the basis of the speckle image and the second parameter value. Then, a speckle index value image is generated on the basis of the calculated speckle index value and displayed on a display unit (74).

### TECHNICAL FIELD

The present disclosure relates to a medical system, an information processing device, and an information processing method.

### BACKGROUND ART

In recent years, in a medical system field, development of a speckle imaging technology capable of constantly observing fluid such as a blood flow, a lymph flow and the like has been advanced. Here, a speckle is a phenomenon in which applied coherent light is reflected by minute irregularities and the like on a surface of a subject (target) and interferes to generate a spotty pattern.

On the basis of this speckle phenomenon, for example, it is possible to discriminate a blood flow portion from a non-blood flow portion in a living body, which is the subject. Specifically, while a speckle contrast value decreases due to motion of red blood cells and the like that reflect the coherent light in the blood flow portion, the speckle contrast value increases because an entire non-blood flow portion is stationary. Therefore, the blood flow portion may be discriminated from the non-blood flow portion on the basis of a speckle contrast image generated using the speckle contrast value of each pixel.

Note that, an index value calculated by performing statistical processing on a luminance value of the speckle (speckle index value) also includes a blur rate (BR), a square BR (SBR), and a mean BR (MBR) in addition to the speckle contrast value. Hereinafter, an image generated using the speckle index value is referred to as a speckle index value image.

In general, an absolute value of the speckle index value corresponding to velocity of fluid (blood and the like) (hereinafter, also referred to as “flow velocity”) and sensitivity (ease of change) of the speckle index value for each flow velocity range are not constant. They change depending on an exposure time of a camera, various parameters (a processing size (5×5 cells and the like), a gain (signal amplification factor) and the like) regarding image processing and the like.

Then, in a case of a subject including both a slow flow velocity portion and a fast flow velocity portion, for example, the speckle index value image in which both the slow flow velocity portion and the fast flow velocity portion are easily visible may be generated by processing images captured with a plurality of exposure times.

### CITATION LIST

### Patent Document

Patent Document 1: Japanese Patent Application Laid-Open No. 2017-170064

### SUMMARY OF THE INVENTION

### Problems to be Solved by the Invention

However, in the conventional speckle imaging technology, it is not possible to easily switch between display in which the slow flow velocity portion is easily visible and display in which the fast flow velocity portion is easily visible in the speckle index value image with a single exposure time. Specifically, in order to perform such switching, it is necessary to individually adjust various parameters, and a work is complicated.

Therefore, the present disclosure proposes a medical system, an information processing device, and an information processing method capable of easily switching between the display in which the slow flow velocity portion is easily visible and the display in which the fast flow velocity portion is easily visible in the speckle index value image with a single exposure time.

### Solutions to Problems

In order to solve the above-described problem, a medical system according to an aspect of the present disclosure is provided with: an irradiation means configured to irradiate a subject with coherent light; an imaging means configured to image reflected light of the coherent light from the subject; an acquisition means configured to acquire a speckle image from the imaging means; a storage means configured to store a first parameter value and a second parameter value different from each other as a parameter for calculating a speckle index value that is a statistical index value for a luminance value of a speckle; a selection means configured to select any one of a first mode corresponding to the first parameter value and a second mode corresponding to the second parameter value; a calculation means configured to calculate the speckle index value on the basis of the speckle image and the first parameter value in a case where the first mode is selected, and to calculate the speckle index value on the basis of the speckle image and the second parameter value in a case where the second mode is selected; a generation means configured to generate a speckle index value image on the basis of the calculated speckle index value; and a display control means configured to allow a display unit to display the speckle index value image.

### BRIEF DESCRIPTION OF DRAWINGS

FIG. 1 is a view illustrating a configuration example of a medical system according to an embodiment of the present disclosure.

FIG. 2 is a view illustrating a configuration example of an information processing device according to the embodiment of the present disclosure.

FIG. 3 is a view illustrating an example of an SC image of a pseudo blood vessel.

FIG. 4 is a graph illustrating a relationship between SC and flow velocity.

FIG. 5 is a flowchart illustrating image processing by the information processing device according to the embodiment of the present disclosure.

FIG. 6 is a view illustrating a first example of a mode selection screen in the embodiment of the present disclosure.

FIG. 7 is a view illustrating a second example of a mode selection screen in the embodiment of the present disclosure.

FIG. 8 is a view schematically illustrating a first example of screen transition in a case where a mode is switched in the embodiment of the present disclosure.

FIG. 9 is a view schematically illustrating a second example of screen transition in a case where a mode is switched in the embodiment of the present disclosure.

FIG. 10 is an explanatory view in a case where a mode in which a dynamic range of the SC of a high flow velocity portion becomes larger is selected in the embodiment of the present disclosure.

FIG. 11 is an explanatory view in a case where a mode in which a dynamic range of the SC of a low flow velocity portion becomes larger is selected in the embodiment of the present disclosure.

FIG. 12 is an explanatory view of a case where various parameter values are adjusted to the blood vessel at low flow velocity and a case where the various parameter values are adjusted to the blood vessel at high flow velocity out of the blood vessels branching into two in the embodiment of the present disclosure.

FIG. 13 is a view schematically illustrating a case where there are two peaks in histogram of the SC in the embodiment of the present disclosure.

FIG. 14 is a view illustrating an example of the SC image obtained by dividing a region into a plurality of regions in the embodiment of the present disclosure.

FIG. 15 is a view illustrating an example of parallel display of a visible light image and the SC image in the embodiment of the present disclosure.

FIG. 16 is a view illustrating an example of a schematic configuration of an endoscopic surgery system according to an application example 1 of the present disclosure.

FIG. 17 is a block diagram illustrating an example of functional configurations of a camera head and a CCU illustrated in FIG. 16.

FIG. 18 is a view illustrating an example of a schematic configuration of a microscopic surgery system according to an application example 2 of the present disclosure.

FIG. 19 is a view illustrating a state of surgery using the microscopic surgery system illustrated in FIG. 18.

### MODE FOR CARRYING OUT THE INVENTION

Hereinafter, an embodiment of the present disclosure is described in detail with reference to the drawings. Note that, in each of the following embodiments, the same components are assigned with the same reference sign, and the description thereof is not repeated appropriately.

First, the purpose of the present invention is described. In a medical field, evaluation of a blood flow is often important. For example, in bypass surgery in brain surgery, patency (blood flow) is confirmed after connecting the blood vessels. Furthermore, in clipping of aneurysm, an inflow of the blood flow into the aneurysm is confirmed after the clipping. In these applications, blood flow evaluation using an ultrasonic Doppler blood flowmeter or by angiography using an indocyanine green (ICG) drug has been performed so far.

However, since the ultrasonic Doppler blood flowmeter measures the blood flow at one point of a portion in contact with a probe, blood flow trend distribution in an entire operative field is unknown. Furthermore, there is a risk that evaluation must be performed by contact with the cerebral blood vessel.

Furthermore, the angiography using the ICG drug utilizes a feature that the ICG drug binds to plasma protein in vivo and emits fluorescence by near-infrared excitation light, and is invasive observation of administering the drug. Furthermore, in terms of the blood flow evaluation, it is necessary to distinguish the flow from a change immediately after administration of the ICG drug, so that there is a limitation in using also in terms of timing.

There is a speckle imaging technology as a blood flow evaluation method for visualizing the blood flow without administering a drug under such circumstances. For example, JP 2017-170064 A discloses an optical device for perfusion evaluation in the speckle imaging technology. Here, a principle of detecting motion (blood flow) using speckles generated by a laser is used. Hereinafter, a case where a speckle contrast is used, for example, as an index of motion detection is described.

The speckle contrast is a value indicated by (standard deviation)/(average value) of light intensity distribution. In a portion without motion, a locally bright portion to a locally dark portion of a speckle pattern are distributed, so that standard deviation of intensity distribution is large and the speckle contrast (degree of glare) is high. In contrast, in a portion with motion, the speckle pattern changes with the motion. Considering that the speckle pattern is imaged by an observation system with a certain exposure time, since the speckle pattern changes in the exposure time, the imaged speckle pattern is averaged, and the speckle contrast (degree of glare) decreases. Especially, the larger the motion, the more the averaging proceeds, so that the speckle contrast decreases. Magnitude of a motion amount may be obtained by evaluating the speckle contrast in this manner.

Then, an absolute value of the speckle contrast (SC (value)) corresponding to velocity of fluid (blood and the like) and sensitivity (ease of change) of the SC for each flow velocity range are not constant. They change depending on an exposure time of a camera, various parameters (a processing size (5×5 cells and the like), a gain (signal amplification factor) and the like) regarding image processing and the like.

Here, FIG. 4 is a graph illustrating a relationship between the SC and the flow velocity. In FIG. 4, the SC is plotted along the ordinate, and the flow velocity is plotted along the abscissa. When the flow velocity is V1, the SC is SC1, and when the flow velocity is V2, the SC is SC2. In a flow velocity range R1 where the flow velocity is lower than the flow velocity V1, the SC changes gradually. Furthermore, in a flow velocity range R3 where the flow velocity is equal to or higher than the flow velocity V2, the SC changes gradually. Then, in a flow velocity range R2 where the flow velocity is equal to or higher than the flow velocity V1 and lower than the flow velocity V2, the SC changes steeply. That is, the sensitivity of the SC in the flow velocity range R2 is high.

Then, in a case of a subject including both a low flow velocity portion and a high flow velocity portion, for example, an SC image in which both the low flow velocity portion and the high flow velocity portion are easily viewable may be generated by processing images captured with a plurality of exposure times.

However, in the conventional speckle imaging technology, it is not possible to easily switch between display in which the low flow velocity portion is easily viewable and display in which the low flow velocity portion is easily viewable in the SC image with a single exposure time. Specifically, in order to perform such switching, it is necessary to individually adjust various parameters, and a work is complicated.

Therefore, in the following, a medical system, an information processing device, and an information processing method capable of easily switching between the display in which the low flow velocity portion is easily viewable and the display in which the high flow velocity portion is easily viewable in the SC image with a single exposure time is described.

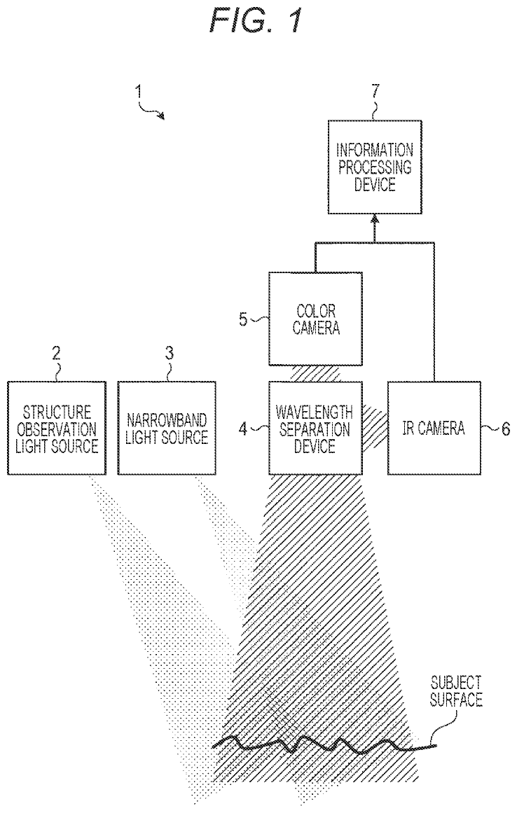

FIG. 1 is a view illustrating a configuration example of a medical system 1 according to an embodiment of the present disclosure. The medical system 1 is provided with a structure observation light source 2 (irradiation means), a narrowband light source 3 (irradiation means), a wavelength separation device 4, a color camera 5 (imaging means), an IR camera 6 (imaging means), and an information processing device 7. Hereinafter, each component is described in detail.

(1) Light Source

The structure observation light source 2 irradiates a subject with incoherent light (for example, incoherent visible light; hereinafter, also simply referred to as “visible light”). Furthermore, the narrowband light source 3 irradiates the subject with coherent light (for example, coherent near-infrared light; hereinafter, also simply referred to as “near infrared light”). Note that, the coherent light refers to light with a phase relationship between light waves at arbitrary two points in a light flux temporally invariable and constant that shows complete coherence even when the light flux is divided by an arbitrary method and then superimposed again with a large optical path difference. Furthermore, the incoherent light refers to light that does not have the above-described properties of the coherent light.

A wavelength of the coherent light output from the narrowband light source 3 according to the present disclosure is preferably, for example, about 800 to 900 nm. For example, when the wavelength is 830 nm, the same optical system as that of ICG observation may be used. That is, since it is common to use near-infrared light with a wavelength of 830 nm in a case where the ICG observation is performed, if near-infrared light with the same wavelength is used for speckle observation, too, the speckle observation may be performed without changing an optical system of a microscope capable of performing the ICG observation.

Note that, the wavelength of the coherent light emitted by the narrowband light source 3 is not limited thereto, and it is assumed that various other wavelengths are used. For example, when the coherent light with a wavelength of 400 to 450 nm (ultraviolet to blue) or the coherent light with a wavelength of 700 to 900 nm (infrared) is used, observation with wavelength separated from that of visible light illumination is easily performed. Furthermore, in a case where visible coherent light with a wavelength of 450 to 700 nm is used, it is easy to select a laser used in a projector and the like. Furthermore, considering an imager other than Si, it is also possible to use the coherent light with a wavelength of 900 nm or longer. Hereinafter, a case where near-infrared light with a wavelength of 830 nm is used as the coherent light is taken as an example.

Furthermore, a type of the narrowband light source 3 that emits the coherent light is not especially limited as long as an effect of the present technology is not impaired. As the narrowband light source 3 that emits laser light, for example, an argon ion (Ar) laser, a helium-neon (He—Ne) laser, a dye laser, a krypton (Cr) laser, a semiconductor laser, a solid-state laser obtained by combining the semiconductor laser and a wavelength conversion optical element and the like may be used alone or in combination.

Note that, the subject is simultaneously irradiated with the visible light from the structure observation light source 2 and the near-infrared light from the narrowband light source 3. Furthermore, a type of the structure observation light source 2 is not especially limited as long as the effect of the present technology is not impaired. As an example, there may be a light emitting diode and the like. Furthermore, as other light sources, there may be a xenon lamp, a metal halide lamp, a high-pressure mercury lamp and the like.

(2) Subject

There may be various subjects, and for example, the subject containing fluid is preferable. Due to the nature of the speckle, there is a property that the speckle is less likely to be generated from the fluid. Therefore, when the subject containing the fluid is imaged using the medical system 1 according to the present disclosure, a boundary between a fluid portion and a non-fluid portion, flow velocity in the fluid portion and the like may be recognized, displayed and the like.

More specifically, for example, the subject may be a living body in which the fluid is blood. For example, by using the medical system 1 according to the present disclosure in microscopic surgery, endoscopic surgery and the like, it is possible to perform surgery while confirming a position of the blood vessel. Therefore, safer and more accurate surgery may be performed, and this may also contribute to further development of medical technology.

(3) Imaging Device

The color camera 5 images reflected light (scattered light) of visible light from the subject. The color camera 5 is, for example, a red/green/blue (RGB) imager for visible light observation.

The IR camera 6 images reflected light (scattered light) of near-infrared light from the subject. The IR camera 6 is, for example, an infrared (IR) imager for speckle observation.

The wavelength separation device 4 is, for example, a dichroic mirror. The wavelength separation device 4 separates received near-infrared light (reflected light) and visible light (reflected light). The color camera 5 captures a visible light image obtained from the visible light separated by the wavelength separation device 4. The IR camera 6 captures a speckle image obtained from the near-infrared light separated by the wavelength separation device 4.

(4) Information Processing Device

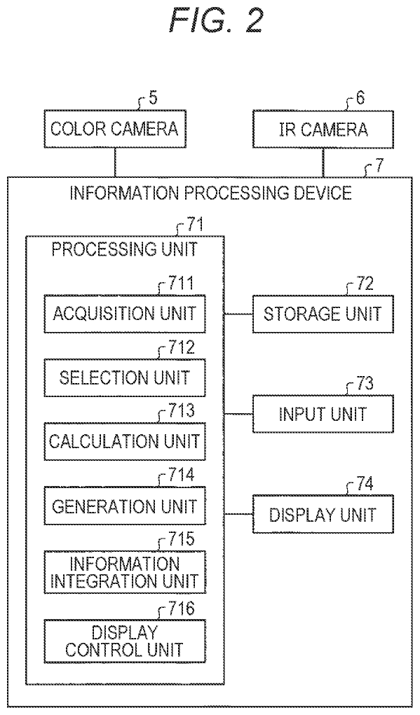

Next, the information processing device 7 is described with reference to FIG. 2. FIG. 2 is a view illustrating a configuration example of the information processing device 7 according to the embodiment of the present disclosure. The information processing device 7 is an image processing device provided with a processing unit 71, a storage unit 72, an input unit 73, and a display unit 74 as main components.

The storage unit 72 stores various types of information such as the visible light image, the speckle image, a calculation result by each unit of the processing unit 71 and the like. Furthermore, the storage unit 72 stores a first parameter value and a second parameter value different from each other as a parameter for calculating the SC that is a statistical index value for a luminance value of the speckle.

The parameter is, for example, a processing size (5×5 cells and the like), a gain (signal amplification factor), an offset, various thresholds (upper limit value and lower limit value), the number of images to be used when temporal noise reduction (NR) is performed and the like. The first parameter value and the second parameter value are set for each parameter (to be described later in detail).

Note that, a storage device outside the medical system 1 may also be used in place of the storage unit 72.

The processing unit 71 is realized by, for example, a central processing unit (CPU), and is provided with an acquisition unit 711 (acquisition means), a selection unit 712 (selection means), a calculation unit 713 (calculation means), a generation unit 714 (generation means), an information integration unit 715, and a display control unit 716 (display control means) as main components.

The acquisition unit 711 acquires the visible light image from the color camera 5 and acquires the speckle image from the IR camera 6. Note that, it is assumed that a position of an imaged subject in the visible light image corresponds to that in the speckle image. That is, in a case where angles of view of both the images coincide with each other, the image may be used as is, and in a case where the angles of view do not coincide with each other, at least one of the images is corrected such that the positions of the imaged subject correspond to each other.

The selection unit 712 selects any one of a first mode corresponding to the first parameter value and a second mode corresponding to the second parameter value. For example, the selection unit 712 selects any one of the first mode and the second mode according to an operation by the user using the calculation unit 713.

For example, the first mode is a mode for observing the blood vessel in which blood flow velocity is relatively high. In this case, the first parameter value is the parameter value corresponding to first velocity assumed as velocity of the fluid in the subject.

Furthermore, for example, the second mode is a mode for observing tissue in which the velocity of the blood flow is relatively low. In this case, the second parameter value is the parameter value corresponding to second velocity lower than the first velocity (also including a case of zero) assumed as the velocity of the fluid in the subject.

Furthermore, the selection unit 712 may select one of the first mode and the second mode in which a dynamic range of the SC in a region of interest of the speckle image is larger.

Furthermore, the selection unit 712 may check histogram distribution of the SC in the region of interest of the speckle image in the first mode and the second mode, and, in a case where there are two peaks, select one with a larger centroid distance between the two peaks. In this case, a plurality of regions of interest may be set in the speckle image.

Furthermore, in addition to the histogram distribution of the SC, the selection unit 712 may select the mode on the basis of, for example, a relative value of the flow velocity (SC) between the regions of interest. Furthermore, for example, the selection unit 712 may select the mode on the basis of information such as a measurement value by a Doppler blood flowmeter and blood pressure information.

Furthermore, in the example described above, there are the two modes of the first mode and the second mode, but there is no limitation. There may be three or more modes. Furthermore, the modes may be hierarchized, and there may be subdivided modes for each of the plurality of modes.

In a case where the first mode is selected, the calculation unit 713 calculates the SC on the basis of the speckle image and the first parameter value, and in a case where the second mode is selected, this calculates the SC on the basis of the speckle image and the second parameter value.

Note that, the speckle contrast value (SC) of an i-th pixel (pixel of interest) may be expressed by following expression (1).

speckle contrast value of i-th pixel=(standard deviation of intensity of i-th pixel and peripheral pixels)/(average of intensities of i-th pixel and peripheral pixels) expression (1)

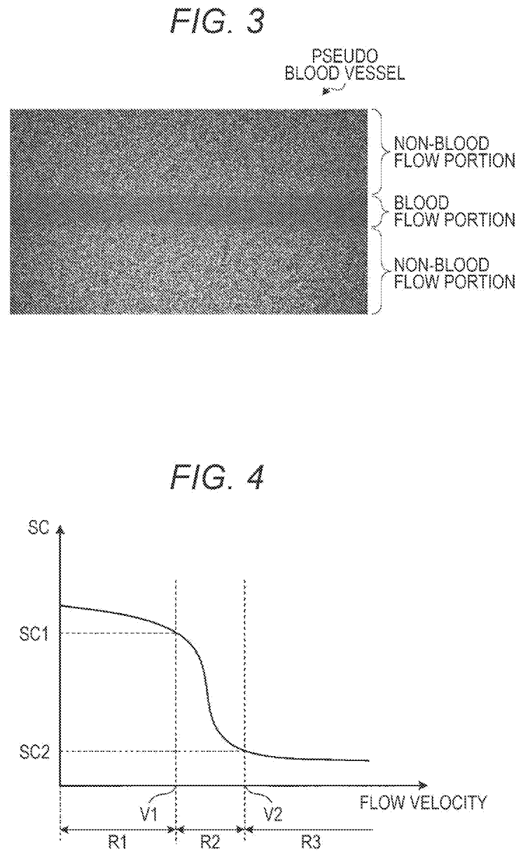

Here, an example of an SC image is described with reference to FIG. 3. FIG. 3 is a view illustrating an example of an SC image of a pseudo blood vessel. As illustrated in the example of the SC image in FIG. 3, many speckles are observed in the non-blood flow portion, and the speckles are scarcely observed in the blood flow portion.

With reference to FIG. 2 again, the generation unit 714 generates the SC image on the basis of the SC calculated by the calculation unit 713. Furthermore, for example, the generation unit 714 discriminates the fluid portion (for example, the blood flow portion) and the non-fluid portion (for example, the non-blood flow portion) on the basis of the SC image. More specifically, for example, the generation unit 714 may discriminate the blood flow portion and the non-blood flow portion by determining whether the SC is equal to or larger than a predetermined SC threshold or not on the basis of the SC image, and may recognize a degree of the blood flow in the blood flow portion.

The information integration unit 715 generates an output image. For example, the information integration unit 715 integrates pieces of information of the SC image and the visible light image.

The display control unit 716 allows the display unit 74 to display an image on the basis of the information integrated by the information integration unit 715. The display control unit 716 allows the display unit 74 to display the SC image and the visible light image in parallel or in a superimposed manner, for example. In this case, the display control unit 716 preferably allows display of the blood flow portion and the non-blood flow portion so as to be distinguishable from each other, for example.

The input unit 73 is a means for inputting information by the user, and is, for example, a keyboard, a mouse, a touch panel and the like.

Under control of the display control unit 716, the display unit 74 displays various types of information such as the visible light image and the speckle image acquired by the acquisition unit 711, the calculation result by each unit of the processing unit 71 and the like. Note that, a display device outside the medical system 1 may be used in place of the display unit 74.

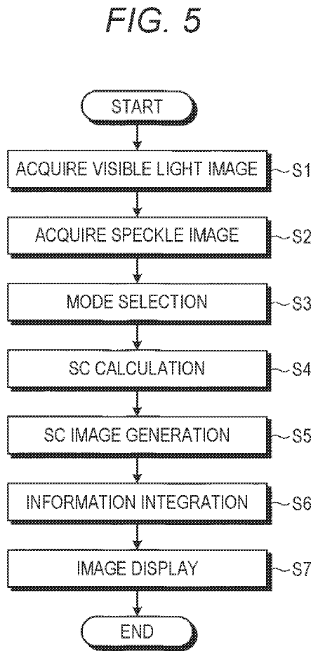

Next, image processing by the information processing device 7 is described with reference to FIG. 5. FIG. 5 is a flowchart illustrating the image processing by the information processing device 7 according to the embodiment of the present disclosure.

First, at step S1, the acquisition unit 711 acquires the visible light image from the color camera 5. Next, at step S2, the acquisition unit 711 acquires the speckle image from the IR camera 6.

Next, at step S3, the selection unit 712 selects any one of the first mode and the second mode according to the operation and the like by the user using the input unit 73.

Next, at step S4, the calculation unit 713 calculates the SC on the basis of the speckle image and the first parameter value in a case where the first mode is selected, and calculates the SC on the basis of the speckle image and the second parameter value in a case where the second mode is selected.

Next, at step S5, the generation unit 714 generates the SC image on the basis of the SC calculated at step S4.

Next, at step S6, the information integration unit 715 integrates the pieces of information of the SC image and the visible light image.

Next, at step S7, the display control unit 716 allows the display unit 74 to display an image on the basis of the information integrated at step S6. The display control unit 716 allows the display unit 74 to display the SC image and the visible light image in parallel or in a superimposed manner, for example. After step S7, the procedure ends.

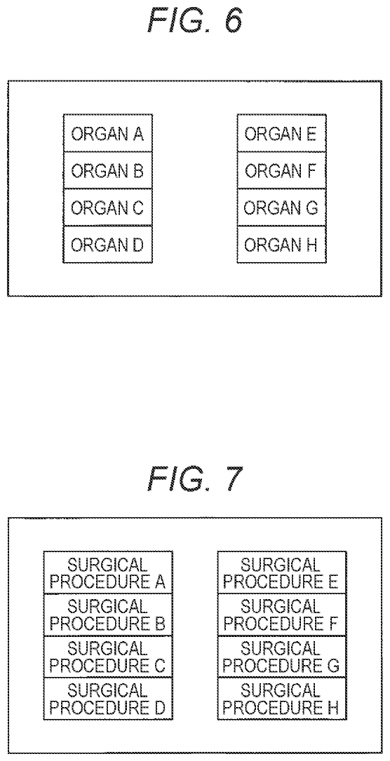

Next, each screen and the like are described with reference to FIGS. 6 to 15. FIG. 6 is a view illustrating a first example of a mode selection screen in the embodiment of the present disclosure. In the mode selection screen illustrated in FIG. 6, eight modes corresponding to organs A to H are prepared. Then, parameter values of various parameters are set for each mode. Therefore, for example, an operator (surgeon who performs surgery) may view the SC image created on the basis of the parameter value suitable for the organ the blood flow in which is desired to be viewed only by selecting any one of the organs A to H on the mode selection screen. Furthermore, in a case where the organ the blood flow in which is desired to be viewed changes, the operator may view the SC image created on the basis of the parameter value suitable for the organ only by newly selecting the mode of the organ desired to be viewed on the mode selection screen as illustrated in FIG. 6.

Next, FIG. 7 is a view illustrating a second example of the mode selection screen in the embodiment of the present disclosure. In the mode selection screen illustrated in FIG. 7, eight modes corresponding to surgical procedures A to H are prepared. Then, parameter values of various parameters are set for each mode. Therefore, for example, the operator may view the SC image created on the basis of the parameter value suitable for the surgical procedure only by selecting any one of the surgical procedures A to H on the mode selection screen. Furthermore, in a case where the surgical procedure changes, the operator may view the SC image created on the basis of the parameter value suitable for the surgical procedure only by newly selecting the mode of a next surgical procedure on the mode selection screen as illustrated in FIG. 7.

As already described with reference to FIG. 4, in the speckle imaging technology, a range of the flow velocity with high sensitivity is narrow, so that a suitable parameter value varies depending on the organ or surgical procedure. Therefore, it is significantly effective from the viewpoint of assisting an intraoperative surgeon that the operator may easily select and switch the mode on the mode selection screen as illustrated in FIGS. 6 and 7.

For example, in clipping of cerebral aneurysm, success or failure of the clipping is confirmed by confirming presence or absence of a minute blood flow.

Furthermore, in cerebral blood vessel bypass surgery, a blood vessel blood flow after connection is confirmed. Furthermore, in resection of brain tumor, there are a scene in which a blood vessel blood flow (fast blood flow) supplying nutrients to the tumor is confirmed and a scene in which a tissue blood flow (slow blood flow) including the periphery is confirmed.

Furthermore, in an operation on the esophagus, there is a procedure in which the stomach made tubular (gastric tube) in place of the resected esophagus is elevated to be anastomosed with the remaining esophagus. Then, in a blood flow confirmation procedure for determining an anastomosis site, it is necessary to confirm the tissue blood flow. Furthermore, in resection and anastomosis of the large intestine, similarly, the tissue blood flow is confirmed to determine the anastomosis site.

Therefore, by preparing the modes suitable for the target organ and the surgical procedure and making it possible to easily select, smooth surgery assistance becomes possible, and safety of surgery is improved. Note that, surgical procedure selection (FIG. 7) may be sub classification after the organ selection (FIG. 6).

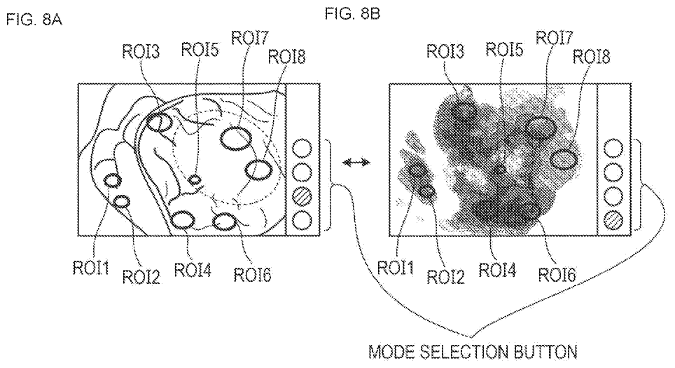

Next, FIG. 8 is a view schematically illustrating a first example of screen transition in a case where the mode is switched in the embodiment of the present disclosure. Even with the same organ, the blood flow desired to be confirmed and required information might be different depending on the procedure. For example, there are a case of focusing on a relatively fast blood vessel blood flow, a case of focusing on a slow tissue blood flow, a case of confirming the presence or absence of the blood flow, a case of observing blood flow velocity distribution and the like. Therefore, modes corresponding to such respective cases are prepared, and a mode selection button for selecting these modes is prepared on the screen as illustrated in FIGS. 8(a) and 8(b). In this case, for example, when the operator selects the mode appropriate to the information that the operator wants regarding any one of regions of interest ROI1 to ROI8, the SC image more suitable for the scene is displayed, so that this is significantly useful. At that time, the visible light image and the SC image may be displayed in a superimposed manner, or may be displayed in parallel by picture-in-picture (PinP) and the like.

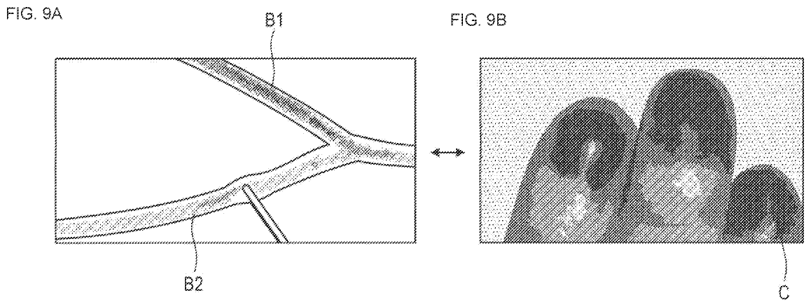

Next, FIG. 9 is a view schematically illustrating a second example of the screen transition in a case where the mode is switched in the embodiment of the present disclosure. FIG. 9(a) illustrates an SC image in a mode of observing the blood vessel blood flow at relatively high blood velocity. In the mode corresponding to the SC image illustrated in FIG. 9(a), the parameter value is set such that the blood flow in a blood vessel B1 out of the blood vessel B1 and a blood vessel B2 after bifurcation is easily viewable.

Furthermore, FIG. 9(b) illustrates an SC image in a mode of observing the tissue blood flow at relatively low blood flow velocity. In the mode corresponding to the SC image illustrated in FIG. 9(b), the parameter value is set such that the tissue blood flow in a fingernail C is easily viewable.

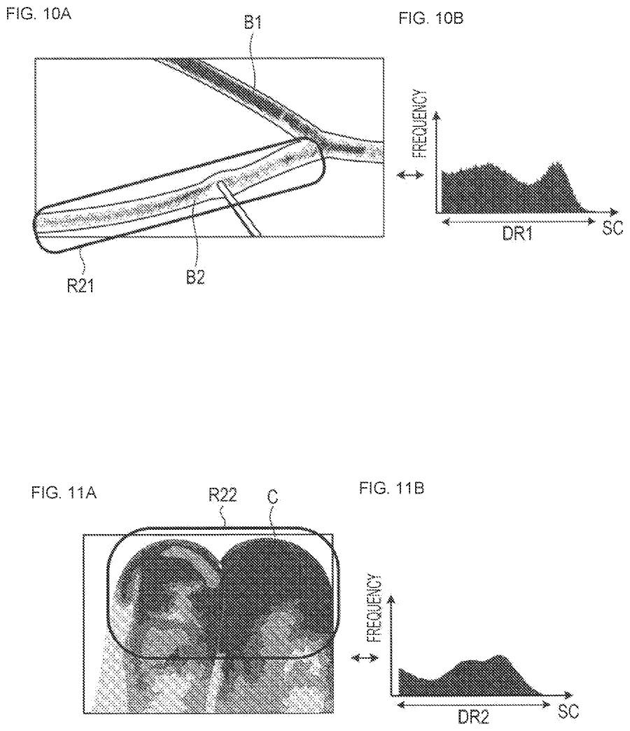

FIG. 10 is an explanatory view in a case where a mode in which a dynamic range of the SC of a high flow velocity portion becomes larger is selected in the embodiment of the present disclosure. An SC image in FIG. 10(a) is the SC image generated in a mode in which a dynamic range (DR1) of the SC of the blood flow portion in the blood vessel B2 in a region R21 becomes larger as illustrated by histogram of the SC in FIG. 10(b). As a result, the SC image in which the blood flow in the blood vessel B2 in the region R21 is easily viewable is displayed, which is useful to the operator.

Furthermore, FIG. 11 is an explanatory view in a case where a mode in which a dynamic range of the SC of a low flow velocity portion becomes larger is selected in the embodiment of the present disclosure. An SC image in FIG. 11(a) is the SC image generated in a mode in which a dynamic range (DR2) of the SC of the blood flow portion in the nail C in a region R22 becomes larger as illustrated by histogram in FIG. 11(b). As a result, the SC image in which the tissue blood flow in the nail C in the region R22 is easily viewable is displayed, which is useful to the operator.

Note that, when describing a magnitude relationship between the parameter values in the mode in FIG. 10 and the mode in FIG. 11, for example, the processing size is smaller in the former, the gain is larger in the former, and the offset is smaller in the former. Note that, there is no limitation, and other magnitude relationships may be employed.

FIG. 12 is an explanatory view of a case where the parameter value is adjusted to the blood vessel at low flow velocity and a case where the parameter value is adjusted to the blood vessel at high flow velocity out of the blood vessels branching into two in the embodiment of the present disclosure. In the histogram of the SC in FIG. 12(a), a region H1 corresponds to a portion without blood flow, a region H2 corresponds to a portion with slow blood flow, and a region H3 corresponds to a portion with fast blood flow.

Then, an SC image displayed so as to widen a color gamut of display of the portion with fast blood flow corresponding to the region H3 is illustrated in FIG. 12(b). In the SC image illustrated in FIG. 12(b), a portion where the blood flow is fast corresponding to the region H3 is the blood vessel B1 in a region R11.

Furthermore, an SC image displayed so as to widen a color gamut of display of the portion with slow blood flow corresponding to the region H2 is illustrated in FIG. 12(c). In the SC image illustrated in FIG. 12(c), a portion where the blood flow is slow corresponding to the region H2 is the blood vessel B2 in a region R12.

In this manner, by appropriately selecting the mode, the operator may easily display the SC image in which flow velocity distribution of the portion where the blood flow is fast is easily visually recognizable as illustrated in FIG. 12(b) or the SC image in which the flow velocity distribution of the portion where the blood flow is slow is easily visually recognizable as illustrated in FIG. 12(c), and this is convenient.

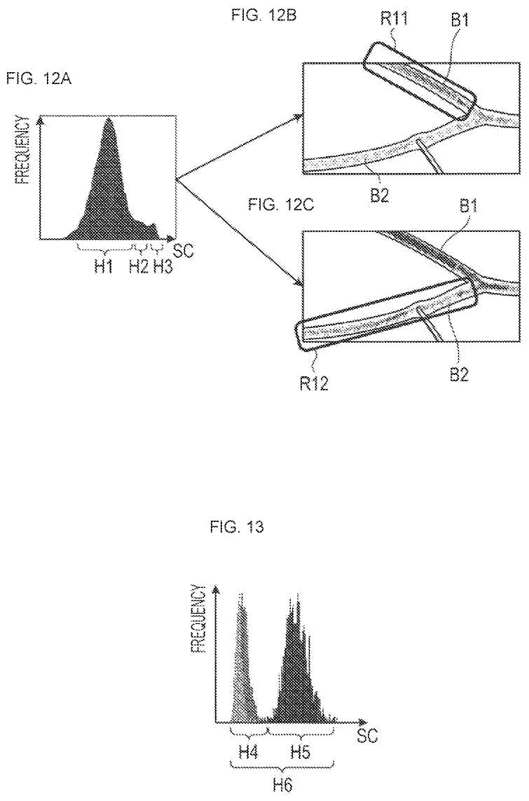

FIG. 13 is a view schematically illustrating a case where there are two peaks in the histogram of the SC in the embodiment of the present disclosure. In the histogram of the SC in FIG. 13, there are two peaks in a region H4 and a region H5. In this case, for example, the selection unit 712 may select a mode in which the dynamic range of the SC in the region H4 becomes larger out of a plurality of modes. In addition, for example, the selection unit 712 may select a mode in which the dynamic range of the SC in the region H5 becomes larger out of a plurality of modes. Furthermore, for example, the selection unit 712 may select a mode in which the dynamic range of the SC in a region H6 obtained by combining the regions H4 and H5 becomes larger out of a plurality of modes.

Furthermore, for example, the selection unit 712 may select a mode in which a centroid distance between the two peaks becomes longer out of a plurality of modes.

In this manner, by appropriately setting a mode selection criterion by the selection unit 712 in advance, it is possible to automatically (that is, without operation by the operator) display the SC image easily viewable by the operator.

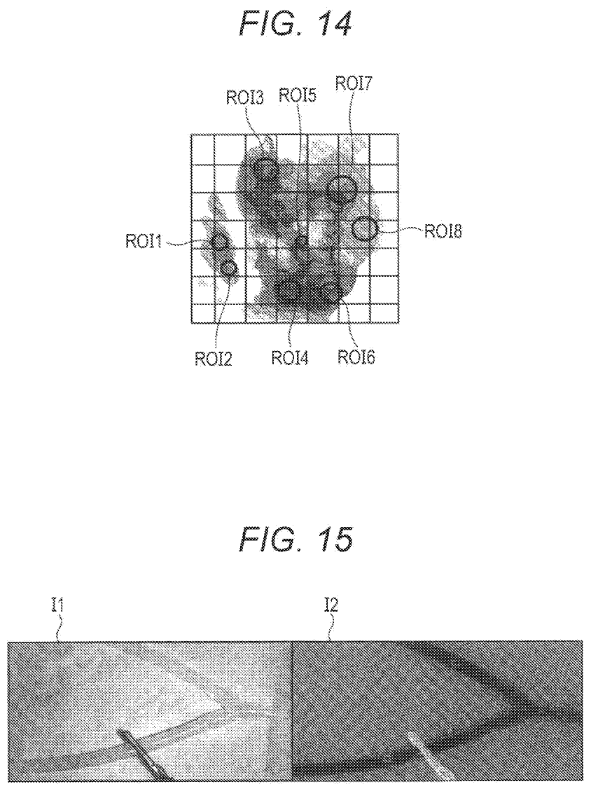

Next, FIG. 14 is a view illustrating an example of the SC image obtained by dividing a region into a plurality of regions in the embodiment of the present disclosure. For example, the selection unit 712 may select a mode for each of the regions of interest ROI1 to ROI8. Alternatively, the selection unit 712 may select a mode for each image region divided in a lattice shape. For the image division, for example, an existing segmentation method may be used. Note that, images captured in different modes may be displayed in parallel or in combination.

FIG. 15 is a view illustrating an example of parallel display of the visible light image and SC image in the embodiment of the present disclosure. As illustrated in FIG. 15, the display control unit 716 may allow the display unit 74 to display an SC image 12 and a visible light image Il in parallel on the basis of the information integrated by the information integration unit 715. Therefore, the operator may appropriately obtain necessary information such as a structure of the subject and the blood flow velocity by viewing both images.

In this manner, according to the medical system 1 of this embodiment, since there is a plurality of modes and the respective modes correspond to the different parameter values, it is possible to easily switch between the display in which the low flow velocity portion is easily viewable and the display in which the high flow velocity portion is easily viewable in the SC image (speckle index value image) with single exposure time only by selecting any mode. Therefore, it is possible to reduce a complication and possibility that the surgeon erroneously diagnose.

Specifically, there is a demand for evaluating the blood flows at various flow velocities in neurosurgery. For example, there is a demand for evaluating, when the blood flow of a certain blood vessel is temporarily stopped, how tissue blood flow around this blood vessel changes. This is an example of determining whether a range in which the blood vessel of interest supplies nutrients is limited only to a tumor or also includes other important functional regions. Then, it is necessary to observe a fast blood flow in order to observe blockage of the blood vessel blood flow, and it is necessary to observe a slow blood flow change in order to observe a change in tissue blood flow. Therefore, a mode switching function in the technology of this embodiment assists in easily performing such a procedure.

Furthermore, as another example in the neurosurgery, for example, there is a demand for evaluating whether, when performing clipping of aneurysm, a penetrating branch around the same is not involved. The penetrating branch is a small blood vessel, but is very important blood vessel that supplies nutrients to a part of the brain. Penetration of the penetrating branch might cause postoperative complications. It is necessary to observe from the fast blood flow (clip start) to the slow blood flow (immediately before clip completion) in order to observe aneurysm blockage, and it is necessary to observe the slow blood flow change in order to observe the blood flow in the penetrating branch. The mode switching function in the technology of this embodiment assists in easily performing such a procedure to avoid the complication.

Furthermore, by displaying the SC image and the visible light image in parallel or in a superimposed manner, it is possible to provide more useful information to the operator.

Furthermore, the selection of the mode may be realized, for example, according to a manual operation by the user. In this manner, the operator may select a mode suitable for a portion of interest in the subject by himself/herself each time and view the SC image created by the mode.

Furthermore, the selection of the mode may be automatically realized on the basis of the dynamic range of the SC, the centroid distance of two peaks in the histogram distribution of the SC and the like. This eliminates the need for a mode selection operation by the operator, which is convenient.

(Application Example 1)

The technology according to the present disclosure may be applied to various products. For example, the technology according to the present disclosure may be applied to an endoscope system. Hereinafter, an endoscopic surgery system as an example of the endoscope system is described.

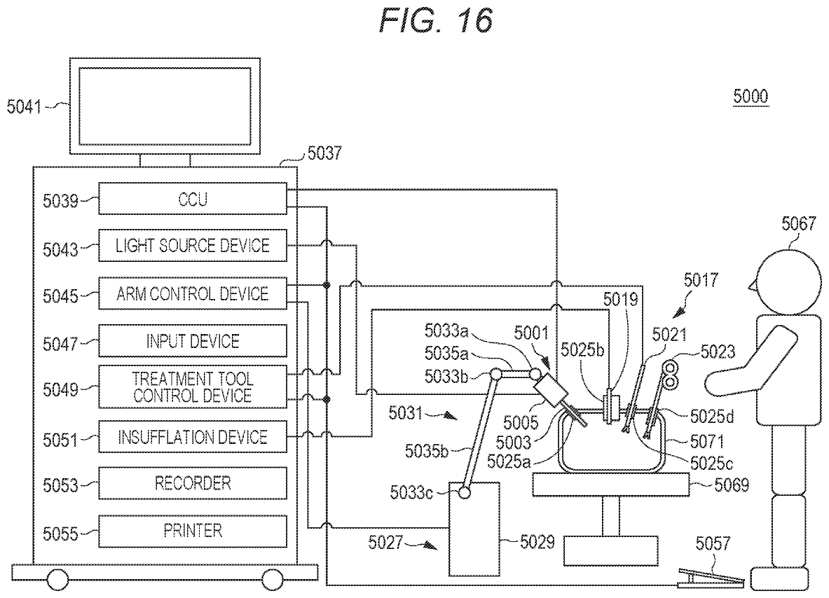

FIG. 16 is a view illustrating an example of a schematic configuration of an endoscopic surgery system 5000 to which the technology according to the present disclosure is applicable. FIG. 16 illustrates a state in which an operator (surgeon) 5067 performs surgery on a patient 5071 on a patient bed 5069 by using the endoscopic surgery system 5000. As illustrated, the endoscopic surgery system 5000 includes an endoscope 5001, other surgical tools 5017, a support arm device 5027 that supports the endoscope 5001, and a cart 5037 on which various devices for endoscopic surgery are mounted.

In the endoscopic surgery, a plurality of tubular hole opening tools referred to as trocars 5025a to 5025d is tapped into the abdominal wall instead of incising the abdominal wall to open the abdomen. Then, a lens tube 5003 of the endoscope 5001 and the other surgical tools 5017 are inserted into the body cavity of the patient 5071 from the trocars 5025a to 5025d. In the illustrated example, an insufflation tube 5019, an energy treatment tool 5021, and forceps 5023 are inserted into the body cavity of the patient 5071 as the other surgical tools 5017. Furthermore, the energy treatment tool 5021 is a treatment tool that performs incision and exfoliation of tissue, sealing of the blood vessel or the like by high-frequency current and ultrasonic vibration. Note that, the illustrated surgical tools 5017 are merely an example, and various surgical tools generally used in the endoscopic surgery, such as tweezers, a retractor and the like, for example, may be used as the surgical tools 5017.

An image of a surgical site in the body cavity of the patient 5071 captured by the endoscope 5001 is displayed on a display device 5041. The operator 5067 performs a procedure such as resection of an affected site, for example, by using the energy treatment tool 5021 and the forceps 5023 while viewing the image of the surgical site displayed on the display device 5041 in real time. Note that, although not illustrated, the insufflation tube 5019, the energy treatment tool 5021, and the forceps 5023 are supported by the operator 5067, an assistant or the like during the surgery.

(Support Arm Device)

The support arm device 5027 is provided with an arm 5031 extending from a base 5029. In the illustrated example, the arm 5031 includes joints 5033a, 5033b, and 5033c, and links 5035a and 5035b, and is driven by control by an arm control device 5045. The arm 5031 supports the endoscope 5001 and controls its position and attitude. Therefore, stable position fixing of the endoscope 5001 may be realized.

(Endoscope)

The endoscope 5001 includes the lens tube 5003 a region of a predetermined length from a distal end of which is inserted into the body cavity of the patient 5071, and a camera head 5005 connected to a proximal end of the lens tube 5003. In the illustrated example, the endoscope 5001 configured as a so-called rigid scope including a rigid lens tube 5003 is illustrated, but the endoscope 5001 may also be configured as a so-called flexible scope including a flexible lens tube 5003.

At the distal end of the lens tube 5003, an opening into which an objective lens is fitted is provided. A light source device 5043 is connected to the endoscope 5001 and light generated by the light source device 5043 is guided to the distal end of the lens tube by a light guide extending inside the lens tube 5003, and applied to an observation target (subject) in the body cavity of the patient 5071 via the objective lens. Note that, the endoscope 5001 may be a forward-viewing endoscope, an oblique-viewing endoscope, or a side-viewing endoscope.

An optical system and an imaging element are provided inside the camera head 5005, and reflected light (observation light) from the observation target is condensed on the imaging element by the optical system. The observation light is photoelectrically converted by the imaging element, and an electric signal corresponding to the observation light, that is, an image signal corresponding to an observation image is generated. The image signal is transmitted as RAW data to a camera control unit (CCU) 5039. Note that, the camera head 5005 has a function of adjusting magnification and a focal distance by appropriately driving the optical system thereof.

Note that, the camera head 5005 may be provided with a plurality of imaging elements in order to support, for example, stereoscopic vision (3D display) and the like. In this case, a plurality of relay optical systems is provided inside the lens tube 5003 in order to guide the observation light to each of the plurality of imaging elements.

(Various Devices Mounted on Cart)

The CCU 5039 includes a central processing unit (CPU), a graphics processing unit (GPU) and the like, and comprehensively controls operations of the endoscope 5001 and the display device 5041. Specifically, the CCU 5039 applies various types of image processing for displaying an image based on the image signal such as, for example, development processing (demosaic processing) to the image signal received from the camera head 5005. The CCU 5039 provides the image signal subjected to the image processing to the display device 5041. Furthermore, the CCU 5039 transmits a control signal to the camera head 5005, and controls drive thereof. The control signal may include information regarding an imaging condition such as the magnification, focal distance and the like.

The display device 5041 displays the image based on the image signal subjected to the image processing by the CCU 5039 under control of the CCU 5039. In a case where the endoscope 5001 supports high-resolution imaging such as 4K (3840 horizontal pixels×2160 vertical pixels), 8K (7680 horizontal pixels×4320 vertical pixels) or the like, and/or supports 3D display, for example, a device capable of performing high-resolution display and/or a device capable of performing 3D display may be used as the display device 5041, respectively, so as to support them. In a case of supporting the high-resolution imaging such as 4K, 8K or the like, by using the display device 5041 having a size of 55 inches or larger, a more immersive feeling may be obtained. Furthermore, a plurality of display devices 5041 having different resolutions and sizes may be provided depending on applications.

The light source device 5043 includes a light source such as, for example, a light emitting diode (LED), and supplies the endoscope 5001 with irradiation light when imaging the surgical site.

The arm control device 5045 includes a processor such as a CPU, for example, and operates according to a predetermined program to control drive of the arm 5031 of the support arm device 5027 according to a predetermined control method.

An input device 5047 is an input interface to the endoscopic surgery system 5000. A user may input various types of information and instructions to the endoscopic surgery system 5000 via the input device 5047. For example, the user inputs various types of information regarding the surgery such as physical information of the patient, information regarding a surgical procedure and the like via the input device 5047. Furthermore, for example, the user inputs an instruction to drive the arm 5031, an instruction to change the imaging condition by the endoscope 5001 (a type of the irradiation light, the magnification, focal distance and the like), an instruction to drive the energy treatment tool 5021 and the like via the input device 5047.

A type of the input device 5047 is not limited, and the input device 5047 may be various well-known input devices. As the input device 5047, for example, a mouse, a keyboard, a touch panel, a switch, a foot switch 5057, and/or a lever may be applied. In a case where the touch panel is used as the input device 5047, the touch panel may be provided on a display surface of the display device 5041.

Alternatively, the input device 5047 is a device worn by the user such as an eyeglass-type wearable device, a head-mounted display (HMD) and the like, for example, and various inputs are performed in accordance with a user's gesture and line-of-sight detected by these devices. Furthermore, the input device 5047 includes a camera capable of detecting motion of the user, and performs various inputs in accordance with the user's gesture and line-of-sight detected from a video imaged by the camera. Moreover, the input device 5047 includes a microphone capable of collecting user's voice, and various inputs are performed by audio via the microphone. In this manner, the input device 5047 is configured to be able to input various types of information in a contactless manner, so that especially the user belonging to a clean area (for example, the operator 5067) may operate a device belonging to an unclean area in a contactless manner. Furthermore, since the user may operate the device without releasing his/her hand from the surgical tool in use, convenience for the user is improved.

A treatment tool control device 5049 controls drive of the energy treatment tool 5021 for cauterization and incision of tissue, sealing of the blood vessel or the like. An insufflation device 5051 injects gas into the body cavity via the insufflation tube 5019 to inflate the body cavity of the patient 5071 for the purpose of securing a visual field by the endoscope 5001 and securing a working space of the operator. A recorder 5053 is a device capable of recording various types of information regarding the surgery. A printer 5055 is a device capable of printing various types of information regarding the surgery in various formats such as text, image, graph or the like.

Hereinafter, a particularly characteristic configuration in the endoscopic surgery system 5000 is described in further detail.

(Support Arm Device)

The support arm device 5027 is provided with the base 5029 as a base, and the arm 5031 extending from the base 5029. In the illustrated example, the arm 5031 includes a plurality of joints 5033a, 5033b, and 5033c, and a plurality of links 5035a and 5035b connected by the joint 5033b, but in FIG. 16, for simplicity, the configuration of the arm 5031 is illustrated in a simplified manner. Actually, shapes, the number, and arrangement of the joints 5033a to 5033c and the links 5035a and 5035b, directions of rotational axes of the joints 5033a to 5033c and the like may be appropriately set so that the arm 5031 has a desired degree of freedom. For example, the arm 5031 may be preferably configured with six or more degrees of freedom. Therefore, since it becomes possible to feely move the endoscope 5001 within a movable range of the arm 5031, the lens tube 5003 of the endoscope 5001 may be inserted into the body cavity of the patient 5071 in a desired direction.

Each of the joints 5033a to 5033c is provided with an actuator, and each of the joints 5033a to 5033c is configured to be rotatable around a predetermined rotational axis by drive of the actuator. The drive of the actuator is controlled by the arm control device 5045, so that rotation angles of the respective joints 5033a to 5033c are controlled, and the drive of the arm 5031 is controlled. Therefore, control of the position and attitude of the endoscope 5001 may be realized. At that time, the arm control device 5045 may control the drive of the arm 5031 by various well-known control methods such as force control, position control or the like.

For example, when the operator 5067 performs an appropriate operation input via the input device 5047 (including a foot switch 5057), the drive of the arm 5031 is appropriately controlled by the arm control device 5045 in accordance with the operation input, and the position and attitude of the endoscope 5001 may be controlled. With this control, it is possible to move the endoscope 5001 at a distal end of the arm 5031 from an arbitrary position to an arbitrary position, and thereafter fixedly support the same in the position after movement. Note that, the arm 5031 may be operated by a so-called master-slave method. In this case, the arm 5031 may be remotely operated by the user via the input device 5047 installed in a location away from an operating room.

Furthermore, in a case where the force control is applied, the arm control device 5045 may perform so-called power assist control of receiving an external force from the user to drive the actuators of the respective joints 5033a to 5033c so that the arm 5031 moves smoothly according to the external force. Therefore, when the user moves the arm 5031 while directly touching the arm 5031, the arm 5031 may be moved with a relatively light force. Therefore, the endoscope 5001 may be moved more intuitively and by a simpler operation, and the user convenience may be improved.

Here, generally, in the endoscopic surgery, the endoscope 5001 has been supported by a surgeon called a scopist. In contrast, by using the support arm device 5027, the position of the endoscope 5001 may be more reliably fixed without manual operation, so that the image of the surgical site may be stably obtained and the surgery may be performed smoothly.

Note that, the arm control device 5045 is not necessarily provided on the cart 5037. Furthermore, the arm control device 5045 is not necessarily one device. For example, the arm control device 5045 may be provided on each of the joints 5033a to 5033c of the arm 5031 of the support arm device 5027, and a plurality of arm control devices 5045 may cooperate with each other to realize drive control of the arm 5031.

(Light Source Device)

The light source device 5043 supplies the endoscope 5001 with the irradiation light when imaging the surgical site. The light source device 5043 includes, for example, a white light source including an LED, a laser light source, or a combination thereof. Since output intensity and output timing of each color (each wavelength) may be controlled with a high degree of accuracy in a case where the white light source is formed by the combination of RGB laser light sources, the light source device 5043 may adjust white balance of the captured image. Furthermore, in this case, by irradiating the observation target with the laser light from each of the RGB laser light sources in time division manner and controlling drive of the imaging element of the camera head 5005 in synchronism with irradiation timing, it is possible to capture images corresponding to RGB in time division manner. According to this method, a color image may be obtained without providing a color filter on the imaging element.

Furthermore, the drive of the light source device 5043 may be controlled such that the intensity of the light to be output is changed every predetermined time. By controlling the drive of the imaging element of the camera head 5005 in synchronization with change timing of the light intensity to obtain the images in time division manner and combining the images, an image of a high dynamic range without so-called black defect and halation may be generated.

Furthermore, the light source device 5043 may be configured to be able to supply light of a predetermined wavelength band corresponding to special light observation. In the special light observation, for example, by using wavelength dependency of absorption of light in body tissue, by applying light of a narrower band than that of the irradiation light (that is, white light) at ordinary observation, so-called narrowband imaging is performed in which predetermined tissue such as the blood vessel in the mucosal surface layer and the like is imaged with high contrast. Alternatively, in the special light observation, fluorescent observation for obtaining an image by fluorescence generated by irradiation of excitation light may be performed. In the fluorescent observation, it is possible to irradiate the body tissue with the excitation light to observe the fluorescence from the body tissue (autonomous fluorescent observation), locally inject a reagent such as indocyanine green (ICG) to the body tissue and irradiate the body tissue with the excitation light corresponding to a fluorescent wavelength of the reagent, thereby obtaining a fluorescent image or the like. The light source device 5043 may be configured to be able to supply the narrowband light and/or excitation light supporting such special light observation.

(Camera Head and CCU)

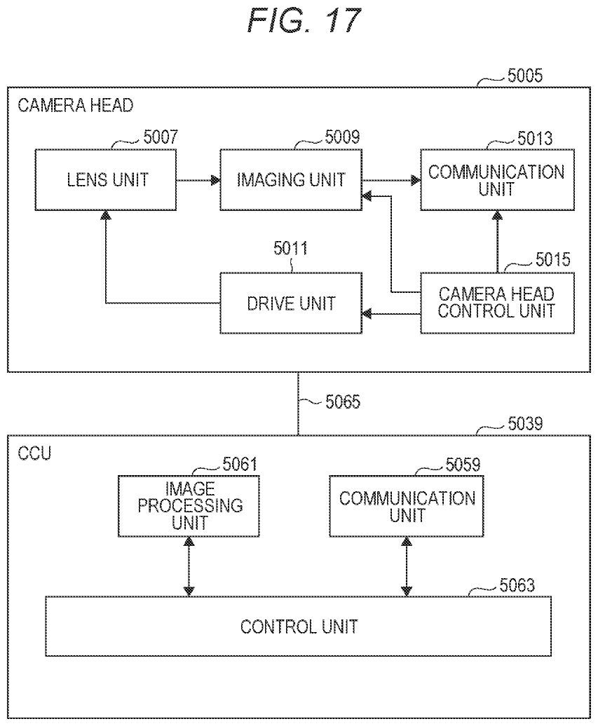

With reference to FIG. 17, functions of the camera head 5005 and the CCU 5039 of the endoscope 5001 are described in further detail. FIG. 17 is a block diagram illustrating an example of functional configurations of the camera head 5005 and the CCU 5039 illustrated in FIG. 16.

With reference to FIG. 17, the camera head 5005 includes a lens unit 5007, an imaging unit 5009, a drive unit 5011, a communication unit 5013, and a camera head control unit 5015 as functions thereof. Furthermore, the CCU 5039 includes a communication unit 5059, an image processing unit 5061, and a control unit 5063 as functions thereof. The camera head 5005 and the CCU 5039 are connected to each other so as to be able to bidirectionally communicate by a transmission cable 5065.

First, a functional configuration of the camera head 5005 is described. The lens unit 5007 is an optical system provided at a connection to the lens tube 5003. The observation light taken in from the distal end of the lens tube 5003 is guided to the camera head 5005 and is incident on the lens unit 5007. The lens unit 5007 is formed by combining a plurality of lenses including a zoom lens and a focus lens. An optical characteristic of the lens unit 5007 is adjusted such that the observation light is condensed on a light-receiving surface of the imaging element of the imaging unit 5009. Furthermore, the zoom lens and the focus lens are configured such that positions thereof on an optical axis are movable for adjusting magnification and focal point of the captured image.

The imaging unit 5009 includes the imaging element, and is arranged on a subsequent stage of the lens unit 5007. The observation light that passes through the lens unit 5007 is condensed on the light-receiving surface of the imaging element, and the image signal corresponding to the observation image is generated by photoelectric conversion. The image signal generated by the imaging unit 5009 is provided to the communication unit 5013.

As the imaging element that forms the imaging unit 5009, for example, a complementary metal oxide semiconductor (CMOS) type image sensor having a Bayer array capable of performing color imaging is used. Note that, as the imaging element, that capable of supporting the imaging of a high-resolution image of, for example, 4K or more may be used. Since the image of the surgical site at high resolution may be obtained, the operator 5067 may grasp the state of the surgical site in further detail, and may proceed with the surgery more smoothly.

Furthermore, the imaging element forming the imaging unit 5009 includes a pair of imaging elements for obtaining image signals for right eye and left eye corresponding to 3D display. By the 3D display, the operator 5067 may grasp a depth of the living tissue in the surgical site more accurately. Note that, in a case where the imaging unit 5009 is of a multiple plate type, a plurality of systems of lens units 5007 is provided so as to correspond to the respective imaging elements.

Furthermore, the imaging unit 5009 is not necessarily provided on the camera head 5005. For example, the imaging unit 5009 may be provided inside the lens tube 5003 immediately after the objective lens.

The drive unit 5011 includes an actuator and moves the zoom lens and the focus lens of the lens unit 5007 by a predetermined distance along the optical axis under control of the camera head control unit 5015. Therefore, the magnification and focal point of the image captured by the imaging unit 5009 may be appropriately adjusted.

The communication unit 5013 includes a communication device for transmitting and receiving various types of information to and from the CCU 5039. The communication unit 5013 transmits the image signal obtained from the imaging unit 5009 as RAW data to the CCU 5039 via the transmission cable 5065. At that time, it is preferable that the image signal be transmitted by optical communication in order to display the captured image of the surgical site with low latency. At the time of surgery, the operator 5067 performs surgery while observing a state of the affected site with the captured image, so that it is required that a moving image of the surgical site be displayed in real time as much as possible for safer and more reliable surgery. In a case where the optical communication is performed, the communication unit 5013 is provided with a photoelectric conversion module that converts an electric signal into an optical signal. The image signal is converted into the optical signal by the photoelectric conversion module, and then transmitted to the CCU 5039 via the transmission cable 5065.

Furthermore, the communication unit 5013 receives the control signal for controlling drive of the camera head 5005 from the CCU 5039. The control signal includes, for example, the information regarding the imaging condition such as information specifying a frame rate of the captured image, information specifying an exposure value at the time of imaging, and/or information specifying the magnification and focal point of the captured image. The communication unit 5013 provides the received control signal to the camera head control unit 5015. Note that, the control signal from the CCU 5039 may also be transmitted by the optical communication. In this case, the communication unit 5013 is provided with a photoelectric conversion module that converts the optical signal into the electric signal, and the control signal is converted into the electric signal by the photoelectric conversion module, and then provided to the camera head control unit 5015.

Note that, the imaging conditions such as the frame rate, exposure value, magnification, focal point and the like described above are automatically set by the control unit 5063 of the CCU 5039 on the basis of the obtained image signal. That is, the endoscope 5001 is equipped with a so-called auto exposure (AE) function, an auto focus (AF) function, and an auto white balance (AWB) function.

The camera head control unit 5015 controls drive of the camera head 5005 on the basis of the control signal from the CCU 5039 received via the communication unit 5013. For example, the camera head control unit 5015 controls the drive of the imaging element of the imaging unit 5009 on the basis of the information specifying the frame rate of the captured image and/or the information specifying the exposure at the time of imaging. Furthermore, for example, the camera head control unit 5015 appropriately moves the zoom lens and the focus lens of the lens unit 5007 via the drive unit 5011 on the basis of the information specifying the magnification and focal point of the captured image. The camera head control unit 5015 may further have a function of storing information for identifying the lens tube 5003 and the camera head 5005.

Note that, by arranging components such as the lens unit 5007, the imaging unit 5009 and the like in a hermetically sealed structure having high airtightness and waterproofness, the camera head 5005 may have resistance to autoclave sterilization.

Next, a functional configuration of the CCU 5039 is described. The communication unit 5059 includes a communication device for transmitting and receiving various types of information to and from the camera head 5005. The communication unit 5059 receives the image signal transmitted from the camera head 5005 via the transmission cable 5065. At that time, as described above, the image signal may be preferably transmitted by the optical communication. In this case, the communication unit 5059 is provided with a photoelectric conversion module that converts an optical signal into an electric signal corresponding to optical communication. The communication unit 5059 provides the image signal converted into the electric signal to the image processing unit 5061.

Furthermore, the communication unit 5059 transmits a control signal for controlling the drive of the camera head 5005 to the camera head 5005. The control signal may also be transmitted by the optical communication.

The image processing unit 5061 applies various types of image processing to the image signal that is the RAW data transmitted from the camera head 5005. The image processing includes, for example, various types of well-known signal processing such as development processing, high image quality processing (such as band enhancement processing, super-resolution processing, noise reduction (NR) processing, and/or camera shake correction processing), and/or scaling processing (electronic zoom processing). Furthermore, the image processing unit 5061 performs wave detection processing on the image signal for performing AE, AF, and AWB.

The image processing unit 5061 includes a processor such as a CPU, a GPU and the like, and the above-described image processing and wave detection processing may be performed by the processor operating according to a predetermined program. Note that, in a case where the image processing unit 5061 includes a plurality of GPUs, the image processing unit 5061 appropriately divides information regarding the image signal, and performs the image processing in parallel by the plurality of GPUs.

The control unit 5063 performs various controls regarding imaging of the surgical site by the endoscope 5001 and display of the captured image. For example, the control unit 5063 generates the control signal for controlling the drive of the camera head 5005. At that time, in a case where the imaging condition is input by the user, the control unit 5063 generates the control signal on the basis of the input by the user. Alternatively, in a case where the endoscope 5001 has the AE function, AF function, and AWB function, the control unit 5063 appropriately calculates optimal exposure value, focal distance, and white balance in accordance with a result of the wave detection processing by the image processing unit 5061 to generate the control signal.

Furthermore, the control unit 5063 allows the display device 5041 to display the image of the surgical site on the basis of the image signal subjected to the image processing by the image processing unit 5061. At that time, the control unit 5063 recognizes various objects in the surgical site image by using various image recognition technologies. For example, the control unit 5063 may detect a shape, a color and the like of an edge of the object included in the surgical site image, thereby recognizing a surgical tool such as forceps, a specific living-body site, bleeding, mist when using the energy treatment tool 5021 and the like. When allowing the display device 5041 to display the image of the surgical site, the control unit 5063 displays various types of surgery assist information in a superimposed manner on the image of the surgical site using a recognition result. The surgery assist information is displayed in a superimposed manner, and presented to the operator 5067, so that it becomes possible to more safely and reliably proceed with the surgery.

The transmission cable 5065 connecting the camera head 5005 and the CCU 5039 is an electric signal cable supporting communication of electric signals, an optical fiber supporting optical communication, or a composite cable thereof.

Here, in the illustrated example, the communication is performed by wire using the transmission cable 5065, but the communication between the camera head 5005 and the CCU 5039 may be performed wirelessly. In a case where the communication between the both is performed wirelessly, it is not necessary to lay the transmission cable 5065 in the operating room, so that a situation in which movement of medical staffs in the operating room is hindered by the transmission cable 5065 may be solved.

An example of the endoscopic surgery system 5000 to which the technology according to the present disclosure may be applied is described above. Note that, the endoscopic surgery system 5000 is herein described as an example, but a system to which the technology according to the present disclosure may be applied is not limited to such an example. For example, the technology according to the present disclosure may be applied to an inspection flexible endoscopic surgery system or a microscopic surgery system to be described hereinafter in an application example 2.

The technology according to the present disclosure may be preferably applied to the endoscope 5001 out of the configuration described above. Specifically, the technology according to the present disclosure may be applied in a case where the blood flow portion and the non-blood flow portion in the image of the surgical site in the body cavity of the patient 5071 captured by the endoscope 5001 are displayed on the display device 5041 so as to be easily visually recognizable. By applying the technology according to the present disclosure to the endoscope 5001, in the speckle imaging technology, it is possible to easily switch between the display in which the low flow velocity portion is easily viewable and the display in which the high flow velocity portion is easily viewable in the SC image with a single exposure time. Therefore, the operator 5067 may view the SC image with high visibility according to the target organ and the surgical procedure in real time on the display device 5041, and may perform surgery more safely.

### (Application Example 2)

Furthermore, the technology according to the present disclosure may be applied to, for example, a microscopic surgery system. Hereinafter, a microscopic surgery system as an example of the microscope system is described. The microscopic surgery system is a system used for so-called microsurgery performed while observing a minute site of a patient under magnification.

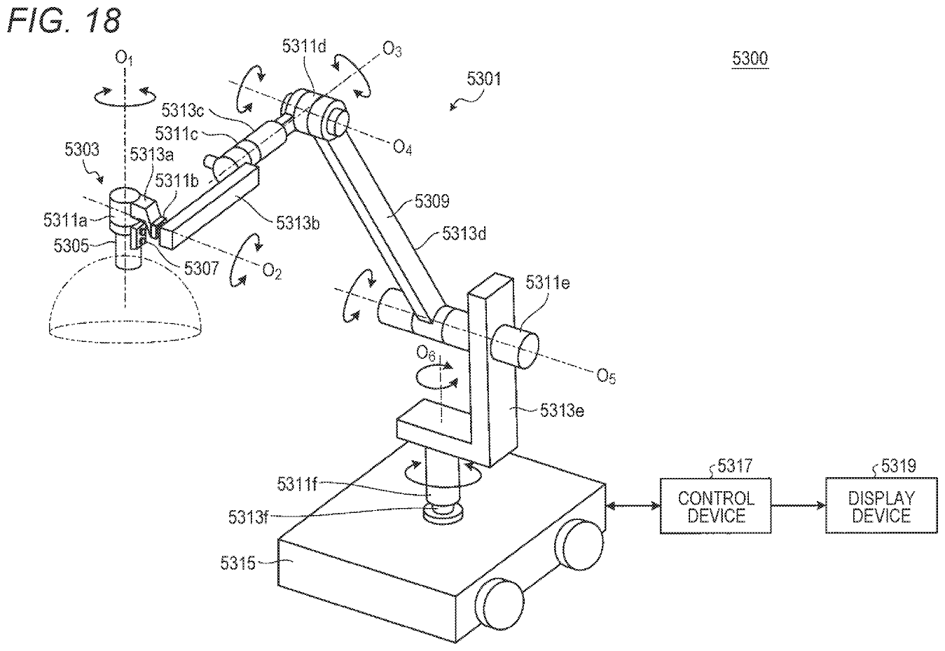

FIG. 18 is a view illustrating an example of a schematic configuration of a microscopic surgery system 5300 to which the technology according to the present disclosure may be applied. With reference to FIG. 18, the microscopic surgery system 5300 includes a microscope device 5301, a control device 5317, and a display device 5319. Note that, in the following description of the microscopic surgery system 5300, a “user” means an arbitrary medical staff who uses the microscopic surgery system 5300 such as an operator, an assistant and the like.

The microscope device 5301 includes a microscope unit 5303 for observing an observation target (surgical site of the patient) under magnification, an arm 5309 that supports the microscope unit 5303 at a distal end thereof, and a base 5315 that supports a proximal end of the arm 5309.

The microscope unit 5303 includes a substantially cylindrical tubular portion 5305, an imaging unit (not illustrated) provided inside the tubular portion 5305, and an operation unit 5307 provided in a partial region on an outer periphery of the tubular portion 5305. The microscope unit 5303 is an electronic imaging microscope unit (so-called video microscope unit) that electronically captures an image by the imaging unit.

A cover glass for protecting the imaging unit inside is provided on an opening surface at a lower end of the tubular portion 5305. Light from the observation target (hereinafter also referred to as observation light) passes through the cover glass to be incident on the imaging unit inside the tubular portion 5305. Note that, a light source of, for example, a light emitting diode (LED) and the like may be provided inside the tubular portion 5305, and at the time of imaging, the observation target may be irradiated with light from the light source via the cover glass.

The imaging unit includes an optical system that condenses the observation light and an imaging element that receives the observation light condensed by the optical system. The optical system is formed by combining a plurality of lenses including a zoom lens and a focus lens, and an optical characteristic thereof is adjusted such that an image of the observation light is formed on a light-receiving surface of the imaging element. The imaging element receives the observation light and photoelectrically converts the same to generate a signal corresponding to the observation light, that is, an image signal corresponding to an observation image. As the imaging element, for example, that including a Bayer array capable of color imaging is used. The imaging element may be various types of well-known imaging elements such as a complementary metal oxide semiconductor (CMOS) image sensor, a charge coupled device (CCD) image sensor or the like. The image signal generated by the imaging element is transmitted to the control device 5317 as RAW data. Here, the transmission of the image signal may be preferably performed by optical communication. At a surgical site, the operator performs surgery while observing a state of an affected site with the captured image, so that it is required that a moving image of the surgical site be displayed in real time as much as possible for safer and more reliable surgical procedure. By transmitting the image signal by the optical communication, the captured image may be displayed with low latency.

Note that, the imaging unit may include a drive mechanism that moves the zoom lens and the focus lens of the optical system along an optical axis. By appropriately moving the zoom lens and the focus lens by the drive mechanism, magnification of the captured image and a focal distance at the time of imaging may be adjusted. Furthermore, the imaging unit may be equipped with various functions that may be generally provided on the electronic imaging microscope unit such as an auto exposure (AE) function, an auto focus (AF) function and the like.

Furthermore, the imaging unit may be configured as a so-called single-plate imaging unit including one imaging element, or may be configured as a so-called multiple-plate imaging unit including a plurality of imaging elements. In a case where the imaging unit is of the multiple-plate type, for example, the image signals corresponding to RGB may be generated by the respective imaging elements, and a color image may be obtained by combining them. Alternatively, the imaging unit may include a pair of imaging elements for obtaining image signals for right eye and left eye corresponding to stereoscopic vision (3D display). By the 3D display, the operator may grasp a depth of living tissue in the surgical site more accurately. Note that, in a case where the imaging unit is of the multiple-plate type, a plurality of optical systems may be provided so as to correspond to the respective imaging elements.

The operation unit 5307 is formed by using, for example, a cross lever, a switch or the like, and is an input means that receives a user operation input. For example, the user may input an instruction to change the magnification of the observation image and the focal distance to the observation target via the operation unit 5307. By appropriately moving the zoom lens and the focus lens by the drive mechanism of the imaging unit according to the instruction, the magnification and focal distance may be adjusted. Furthermore, for example, the user may input an instruction to switch an operation mode (all-free mode and fixed mode to be described later) of the arm 5309 via the operation unit 5307. Note that, in a case where the user wants to move the microscope unit 5303, a mode is assumed in which the user moves the microscope unit 5303 in a state where the user grips the tubular portion 5305. Therefore, the operation unit 5307 is preferably provided in a position where the user may easily operate the same with a finger while gripping the tubular portion 5305 such that the user may operate the same while moving the tubular portion 5305.

The arm 5309 includes a plurality of links (first link 5313a to sixth link 5313f) being rotatably connected to each other by a plurality of joints (first joint 5311a to sixth joint 5311f).

The first joint 5311a has a substantially cylindrical shape, and supports at a distal end (lower end) thereof an upper end of the tubular portion 5305 of the microscope unit 5303 so as to be rotatable around a rotational axis (first axis O1) parallel to a central axis of the tubular portion 5305. Here, the first joint 5311a may be configured such that the first axis O1 coincides with the optical axis of the imaging unit of the microscope unit 5303. Therefore, by rotating the microscope unit 5303 around the first axis O1, it becomes possible to change a visual field so as to rotate the captured image.

The first link 5313a fixedly supports the first joint 5311a at a distal end thereof. Specifically, the first link 5313a is a rod-shaped member having a substantially L shape, and while one side on the distal end side thereof extends in a direction orthogonal to the first axis O1, an end of the one side is connected to the first joint 5311a so as to abut an upper end of an outer periphery of the first joint 5311a. The second joint 5311b is connected to an end of the other side on a proximal end side of the substantially L shape of the first link 5313a.

The second joint 5311b has a substantially cylindrical shape and supports the proximal end of the first link 5313a so as to be rotatable around a rotational axis (second axis O2) orthogonal to the first axis O1. A distal end of the second link 5313b is fixedly connected to a proximal end of the second joint 5311b.

The second link 5313b is a rod-shaped member having a substantially L shape, and while one side on the distal end side thereof extends in a direction orthogonal to the second axis O2, an end of the one side is fixedly connected to the proximal end of the second joint 5311b.

The third joint 5311c is connected to the other side on a proximal end side of the substantially L shape of the second link 5313b.

The third joint 5311c has a substantially cylindrical shape and supports the proximal end of the second link 5313b so as to be rotatable around a rotational axis (third axis O3) orthogonal to the first and second axes O1 and O2 at a distal end thereof. A distal end of the third link 5313c is fixedly connected to a proximal end of the third joint 5311c. The microscope unit 5303 may be moved so as to change a position of the microscope unit 5303 in a horizontal plane by rotating the configuration on the distal end side including the microscope unit 5303 around the second axis O2 and the third axis O3. That is, by controlling the rotation around the second axis O2 and the third axis O3, the visual field of the captured image may be moved in the plane.

The third link 5313c is configured such that the distal end side thereof has a substantially cylindrical shape, and the proximal end of the third joint 5311c is fixedly connected to the distal end of the cylindrical shape such that central axes of both of them are substantially the same. A proximal end side of the third link 5313c has a prismatic shape, and the fourth joint 5311d is connected to an end thereof.

The fourth joint 5311d has a substantially cylindrical shape, and supports the proximal end of the third link 5313c at a distal end thereof so as to be rotatable around a rotational axis (fourth axis O4) orthogonal to the third axis O3. A distal end of the fourth link 5313d is fixedly connected to a proximal end of the fourth joint 5311d.

The fourth link 5313d is a rod-shaped member extending substantially linearly, and while extending so as to be orthogonal to the fourth axis O4, an end on the distal end thereof is fixedly connected to the fourth joint 5311d so as to abut a substantially cylindrical side surface of the fourth joint 5311d. The fifth joint 5311e is connected to a proximal end of the fourth link 5313d.

The fifth joint 5311e has a substantially cylindrical shape, and supports at a distal end side thereof the proximal end of the fourth link 5313d so as to be rotatable around a rotational axis (fifth axis O5) parallel to the fourth axis O4. A distal end of the fifth link 5313e is fixedly connected to a proximal end of the fifth joint 5311e. The fourth axis O4 and the fifth axis O5 are the rotational axes capable of moving the microscope unit 5303 in an up-down direction. By rotating the configuration on the distal end side including the microscope unit 5303 around the fourth axis O4 and the fifth axis O5, a height of the microscope unit 5303, that is, a distance between the microscope unit 5303 and the observation target may be adjusted.

The fifth link 5313e is formed by combining a first member having a substantially L shape in which one side extends in a vertical direction and the other side extends in a horizontal direction, and a second member in a rod shape extending vertically downward from a portion extending horizontally of the first member. The proximal end of the fifth joint 5311e is fixedly connected in the vicinity of an upper end of the portion extending vertically of the first member of the fifth link 5313e. The sixth joint 5311f is connected to a proximal end (lower end) of the second member of the fifth link 5313e.

The sixth joint 5311f has a substantially cylindrical shape and supports at a distal end side thereof the proximal end of the fifth link 5313e so as to be rotatable around a rotational axis (sixth axis O6) parallel to the vertical direction. A distal end of the sixth link 5313f is fixedly connected to a proximal end of the sixth joint 5311f.

The sixth link 5313f is a rod-shaped member extending in the vertical direction, and the proximal end thereof is fixedly connected to an upper surface of the base 5315.

A rotatable range of the first joint 5311a to the sixth joint 5311f is appropriately set such that the microscope unit 5303 may move desirably. Therefore, in the arm 5309 having the above-described configuration, motion of total of six-degree freedom of translational three-degree freedom and rotational three-degree freedom may be realized regarding the motion of the microscope unit 5303. In this manner, by configuring the arm 5309 such that the six-degree freedom is realized regarding the movement of the microscope unit 5303, it is possible to freely control the position and attitude of the microscope unit 5303 within the movable range of the arm 5309. Therefore, the surgical site may be observed from any angle, and the surgery may be performed more smoothly.

Note that, the configuration of the arm 5309 illustrated is merely an example, and the number and shapes (lengths) of the links forming the arm 5309, the number and arranged positions of the joints, the directions of the rotational axes and the like may be appropriately designed such that a desired degree of freedom may be realized. For example, as described above, in order to freely move the microscope unit 5303, the arm 5309 is preferably configured with the six-degree freedom, but the arm 5309 may also be configured with a larger degree of freedom (that is, a redundant degree of freedom). In a case where there is the redundant degree of freedom, the arm 5309 may change the attitude of the arm 5309 in a state in which the position and attitude of the microscope unit 5303 are fixed. Therefore, for example, control that is more convenient for the operator may be realized, such as control of the attitude of the arm 5309 so that the arm 5309 does not interfere with an eyesight of the operator who looks at the display device 5319 and the like.

Here, each of the first joint 5311a to the sixth joint 5311f may be provided with an actuator equipped with a drive mechanism such as a motor, an encoder that detects a rotation angle at each joint and the like. Then, drive of each actuator provided on the first joint 5311a to the sixth joint 5311f is appropriately controlled by the control device 5317, so that the attitude of the arm 5309, that is, the position and attitude of the microscope unit 5303 may be controlled. Specifically, the control device 5317 may grasp current attitude of the arm 5309 and current position and attitude of the microscope unit 5303 on the basis of information regarding the rotation angle of each joint detected by the encoder. The control device 5317 calculates a control value (for example, rotation angle, generated torque or the like) for each joint that realizes movement of the microscope unit 5303 according to the operation input from the user by using the grasped information, and drives the drive mechanism of each joint according to the control value. Note that, at that time, a control method of the arm 5309 by the control device 5317 is not limited, and various well-known control methods such as force control, position control or the like may be applied.

For example, when the operator appropriately performs the operation input via an input device not illustrated, the drive of the arm 5309 may be appropriately controlled by the control device 5317 in accordance with the operation input, and the position and attitude of the microscope unit 5303 may be controlled. With this control, it is possible to move the microscope unit 5303 from an arbitrary position to an arbitrary position, and fixedly support this in the position after movement. Note that, as for the input device, in consideration of the convenience of the operator, it is preferable to apply the one that may be operated even if the operator has a surgical tool in his/her hand such as, for example, a foot switch. Furthermore, a contactless operation input may be performed on the basis of gesture detection or line-of-sight detection using a wearable device or a camera provided in an operating room. Therefore, even a user belonging to a clean area may operate a device belonging to an unclean area with a higher degree of freedom. Alternatively, the arm 5309 may be operated in a so-called master slave method. In this case, the arm 5309 may be remotely operated by the user via an input device installed in a place away from the operating room.

Furthermore, in a case where the force control is applied, so-called power assist control of receiving an external force from the user to drive the actuators of the first to sixth joints 5311a to 5311f so that the arm 5309 moves smoothly according to the external force may be performed. Therefore, when the user grips the microscope unit 5303 to directly move the position thereof, the microscope unit 5303 may be moved with a relatively light force. Therefore, the microscope unit 5303 may be moved more intuitively and with a simpler operation, and user convenience may be improved.

Furthermore, the drive of the arm 5309 may be controlled so as to perform a pivot operation. Here, the pivot operation is an operation of moving the microscope unit 5303 so that the optical axis of the microscope unit 5303 is always directed to a predetermined point in space (hereinafter referred to as a pivot point). According to the pivot operation, the same observation position may be observed in various directions, so that observation of the affected site in further detail becomes possible. Note that, in a case where the microscope unit 5303 is configured so as not to be able to adjust a focal distance thereof, it is preferable that the pivot operation is performed in a state in which a distance between the microscope unit 5303 and the pivot point is fixed. In this case, the distance between the microscope unit 5303 and the pivot point may be adjusted to a fixed focal distance of the microscope unit 5303. Therefore, the microscope unit 5303 moves on a hemisphere (schematically illustrated in FIG. 18) having a radius corresponding to the focal distance centered on the pivot point, and a sharp captured image may be obtained even when the observation direction is changed. In contrast, in a case where the microscope unit 5303 is configured to be able to adjust the focal distance thereof, it is possible that the pivot operation is performed in a state in which the distance between the microscope unit 5303 and the pivot point is variable. In this case, for example, the control device 5317 may calculate the distance between the microscope unit 5303 and the pivot point on the basis of information regarding the rotation angle of each joint detected by the encoder, and automatically adjust the focal distance of the microscope unit 5303 on the basis of a calculation result. Alternatively, in a case where the microscope unit 5303 has an AF function, the focal distance may be automatically adjusted by the AF function every time the distance between the microscope unit 5303 and the pivot point is changed by the pivot operation.

Furthermore, each of the first joint 5311a to the sixth joint 5311f may be provided with a brake that restricts the rotation thereof. The operation of the brake may be controlled by the control device 5317. For example, in a case where it is desired to fix the position and attitude of the microscope unit 5303, the control device 5317 activates the brake of each joint. Therefore, since the attitude of the arm 5309, that is, the position and attitude of the microscope unit 5303 may be fixed without driving the actuator, power consumption may be reduced. In a case where it is desired to move the position and attitude of the microscope unit 5303, the control device 5317 is only required to release the brake of each joint and drive the actuator according to a predetermined control method.

Such a brake operation may be performed in response to an operation input by the user via the operation unit 5307 described above. In a case where the user wants to move the position and attitude of the microscope unit 5303, the user operates the operation unit 5307 to release the brake of each joint. Therefore, the operation mode of the arm 5309 shifts to a mode (all-free mode) in which the rotation at each joint may be freely performed. Furthermore, in a case where the user wants to fix the position and attitude of the microscope unit 5303, the user operates the operation unit 5307 to activate the brake of each joint. Therefore, the operation mode of the arm 5309 shifts to a mode (fixed mode) in which the rotation at each joint is restricted.

The control device 5317 comprehensively controls the operation of the microscopic surgery system 5300 by controlling the operations of the microscope device 5301 and the display device 5319. For example, the control device 5317 controls the drive of the arm 5309 by operating the actuators of the first joint 5311a to the sixth joint 5311f according to a predetermined control method. Furthermore, for example, the control device 5317 changes the operation mode of the arm 5309 by controlling the operation of the brake of the first joint 5311a to the sixth joint 5311f. Furthermore, for example, the control device 5317 generates image data for display by applying various types of signal processing to the image signal obtained by the imaging unit of the microscope unit 5303 of the microscope device 5301 and allows the display device 5319 to display the image data. As the signal processing, for example, various types of well-known signal processing such as development processing (demosaic processing), high image quality processing (such as band enhancement processing, super-resolution processing, noise reduction (NR) processing, and/or camera shake correction processing), and/or scaling processing (that is, electronic zoom processing) may be performed.

Note that, communication between the control device 5317 and the microscope unit 5303 and communication between the control device 5317 and the first joint 5311a to the sixth joint 5311f may be wired communication or wireless communication. In a case of the wired communication, communication using electric signals may be performed, or optical communication may be performed. In this case, a transmission cable used for the wired communication may be configured as an electric signal cable, an optical fiber, or a composite cable thereof depending on a communication method. In contrast, in a case of the wireless communication, it is not necessary to lay the transmission cable in the operating room, so that a situation in which movement of medical staffs in the operating room is hindered by the transmission cable may be solved.

The control device 5317 may be a microcomputer, a control board or the like on which a processor such as a central processing unit (CPU), a graphics processing unit

(GPU) and the like are mounted, or the processor and a storage element such as a memory are mixedly mounted. The various functions described above may be realized by the processor of the control device 5317 operating according to a predetermined program. Note that, in the illustrated example, the control device 5317 is provided as a separate device from the microscope device 5301; however, the control device 5317 may be installed inside the base 5315 of the microscope device 5301 to be integrated with the microscope device 5301.

Alternatively, the control device 5317 may include a plurality of devices. For example, it is possible that a microcomputer, a control board and the like are arranged on each of the microscope unit 5303 and the first joint 5311a to the sixth joint 5311f of the arm 5309, and they are connected so as to be able to communicate with each other, so that a function similar to that of the control device 5317 is realized.

The display device 5319 is provided in the operating room, and displays an image corresponding to the image data generated by the control device 5317 under control of the control device 5317. That is, the display device 5319 displays an image of the surgical site captured by the microscope unit 5303. Note that, the display device 5319 may display various types of information regarding the surgery such as physical information of the patient, information regarding a surgical procedure and the like, for example, in place of or together with the image of the surgical site. In this case, the display of the display device 5319 may be appropriately switched by an operation by the user. Alternatively, a plurality of display devices 5319 may be provided, and each of a plurality of display devices 5319 may display the image of the surgical site and various types of information regarding the surgery. Note that, as the display device 5319, various well-known display devices such as a liquid crystal display device, an electro luminescence (EL) display device and the like may be applied.

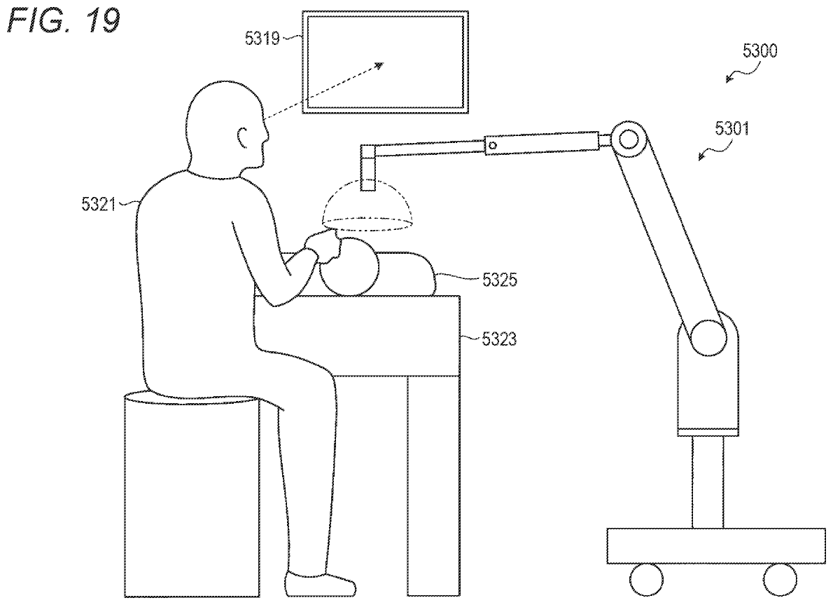

FIG. 19 is a view illustrating a state of surgery using the microscopic surgery system 5300 illustrated in FIG. 18. FIG. 19 schematically illustrates a state in which an operator 5321 performs surgery on a patient 5325 on a patient bed 5323 by using the microscopic surgery system 5300. Note that, in FIG. 19, for simplicity, the control device 5317 out of the configuration of the microscopic surgery system 5300 is not illustrated, and the microscope device 5301 is illustrated in a simplified manner.

As illustrated in FIG. 2C, at the time of surgery, the image of the surgical site captured by the microscope device 5301 is displayed in an enlarged manner on the display device 5319 installed on a wall surface of the operating room using the microscopic surgery system 5300.

The display device 5319 is installed in a position facing the operator 5321, and the operator 5321 performs various procedures on the surgical site such as resection of the affected site, for example, while observing the state of the surgical site by a video displayed on the display device 5319.

An example of the microscopic surgery system 5300 to which the technology according to the present disclosure may be applied is described above. Note that, the microscopic surgery system 5300 is herein described as an example, but a system to which the technology according to the present disclosure may be applied is not limited to such an example. For example, the microscope device 5301 may serve as a support arm device that supports another observation device or another surgical tool in place of the microscope unit 5303 at the distal end thereof. As another observation device described above, for example, an endoscope may be applied. Furthermore, as another surgical tool described above, forceps, tweezers, an insufflation tube for insufflation, an energy treatment tool for incising tissue or sealing the blood vessel by cauterization or the like may be applied. By supporting such observation device and surgical tool with the support arm device, it is possible to fix the position more stably and reduce a burden on the medical staff as compared to a case where the medical staff supports the same manually. The technology according to the present disclosure may be applied to the support arm device that supports such configuration other than the microscope unit.

The technology according to the present disclosure may be preferably applied to the control device 5317 out of the configuration described above. Specifically, the technology according to the present disclosure may be applied in a case where the blood flow portion and the non-blood flow portion in the image of the surgical site of the patient 5325 captured by the imaging unit of the microscope unit 5303 are displayed on the display device 5319 so as to be easily visually recognizable. By applying the technology according to the present disclosure to the control device 5317, in the speckle imaging technology, it is possible to easily switch between the display in which the low flow velocity portion is easily viewable and the display in which the high flow velocity portion is easily viewable in the SC image with a single exposure time. Therefore, the operator 5321 may view the SC image with high visibility according to the target organ and the surgical procedure in real time on the display device 5319, and may perform surgery more safely.

Note that, the present technology may also have following configurations.

(1)

A medical system provided with:

an irradiation means configured to irradiate a subject with coherent light;

an imaging means configured to image reflected light of the coherent light from the subject;

an acquisition means configured to acquire a speckle image from the imaging means;

a storage means configured to store a first parameter value and a second parameter value different from each other as a parameter for calculating a speckle index value that is a statistical index value for a luminance value of a speckle;

a selection means configured to select any one of a first mode corresponding to the first parameter value and a second mode corresponding to the second parameter value;

a calculation means configured to calculate the speckle index value on the basis of the speckle image and the first parameter value in a case where the first mode is selected, and to calculate the speckle index value on the basis of the speckle image and the second parameter value in a case where the second mode is selected;

a generation means configured to generate a speckle index value image on the basis of the calculated speckle index value; and

a display control means configured to allow a display unit to display the speckle index value image.

(2)

The medical system according to (1), in which the medical system is a microscope system or an endoscope system.

(3)

An information processing device provided with:

an acquisition means configured to acquire a speckle image from an imaging means that images reflected light of coherent light with which a subject is irradiated;

a storage means configured to store a first parameter value and a second parameter value different from each other as values of a parameter for calculating a speckle index value that is a statistical index value for a luminance value of a speckle;

a selection means configured to select any one of a first mode corresponding to the first parameter value and a second mode corresponding to the second parameter value;

a calculation means configured to calculate the speckle index value on the basis of the speckle image and the first parameter value in a case where the first mode is selected, and to calculate the speckle index value on the basis of the speckle image and the second parameter value in a case where the second mode is selected;

a generation means configured to generate a speckle index value image on the basis of the calculated speckle index value; and

a display control means configured to allow a display unit to display the speckle index value image.

(4)

The information processing device according to (3), in which

the first parameter value is a parameter value corresponding to first velocity assumed as velocity of fluid in the subject, and

the second parameter value is a parameter value corresponding to second velocity lower than the first velocity assumed as velocity of fluid in the subject.

(5)

The information processing device according (3), in which

the acquisition means further acquires a visible light image from an imaging means that images reflected light of incoherent visible light with which the subject is irradiated, and

the display control means allows the display unit to display the speckle index value image and the visible light image in parallel or in a superimposed manner.

(6)

The information processing device according to (3), in which the selection means selects any one of the first mode and the second mode according to an operation by a user.

(7)

The information processing device according to (3), in which the selection means selects one of the first mode and the second mode in which a dynamic range of the speckle index value in a region of interest of the speckle image is larger.

(8)

The information processing device according to (3), in which the selection means checks histogram distribution of the speckle index value in a region of interest of the speckle image in the first mode and the second mode, and, in a case where there are two peaks, selects one of the modes in which a centroid distance between the two peaks is larger.

(9)

An information processing method using a first parameter value and a second parameter value different from each other as values of a parameter for calculating a speckle index value that is a statistical index value for a luminance value of a speckle, the method provided with:

an acquisition step of acquiring a speckle image from an imaging means that images reflected light of coherent light with which a subject is irradiated;

a selection step of selecting any one of a first mode corresponding to the first parameter value and a second mode corresponding to the second parameter value;

a calculation step of calculating the speckle index value on the basis of the speckle image and the first parameter value in a case where the first mode is selected, and calculating the speckle index value on the basis of the speckle image and the second parameter value in a case where the second mode is selected;

a generation step of generating a speckle index value image on the basis of the calculated speckle index value; and

a display control step of allowing a display unit to display the speckle index value image.

Although the embodiments and variation of the present disclosure are described above, the technical scope of the present disclosure is not limited to the above-described embodiments and variation, and various modifications may be made without departing from the gist of the present disclosure. Furthermore, the components of different embodiments and variation may be appropriately combined.

Note that, the effects in each embodiment and variation of this specification are illustrative only and are not limitative; there may also be another effect.

Furthermore, in each of the above-described embodiments, the speckle contrast value (SC) is described as an example of the statistical index value of the luminance value of the speckle, but there is no limitation and this may be a blur rate (BR), a square BR (SBR), a mean BR (MBR and the like.

Furthermore, in the above-described embodiment, the time of intraoperative assistance is mainly described, but there is no limitation. The present invention may also be applied to image analysis after surgery by storing intraoperative speckle images.

Furthermore, in the image processing illustrated in FIG. 5, other processing such as noise correction may be performed.

### REFERENCE SIGNS LIST

1 Medical system

2 Structure observation light source

3 Narrowband light source

4 Wavelength separation device

5 Color camera

6 IR camera

7 Information processing device

71 Processing unit

72 Storage unit

73 Input unit

74 Display unit

711 Acquisition unit

712 Selection unit

713 Calculation unit

714 Generation unit

715 Information integration unit

716 Display control unit

1. A medical system comprising:
an irradiation means configured to irradiate a subject with coherent light;
an imaging means configured to image reflected light of the coherent light from the subject;
an acquisition means configured to acquire a speckle image from the imaging means;
a storage means configured to store a first parameter value and a second parameter value different from each other as a parameter for calculating a speckle index value that is a statistical index value for a luminance value of a speckle;
a selection means configured to select any one of a first mode corresponding to the first parameter value and a second mode corresponding to the second parameter value;
a calculation means configured to calculate the speckle index value on a basis of the speckle image and the first parameter value in a case where the first mode is selected, and to calculate the speckle index value on a basis of the speckle image and the second parameter value in a case where the second mode is selected;
a generation means configured to generate a speckle index value image on a basis of the calculated speckle index value; and
a display control means configured to allow a display unit to display the speckle index value image.

2. The medical system according to claim 1, wherein the medical system is a microscope system or an endoscope system.

3. An information processing device comprising:
an acquisition means configured to acquire a speckle image from an imaging means that images reflected light of coherent light with which a subject is irradiated;
a storage means configured to store a first parameter value and a second parameter value different from each other as values of a parameter for calculating a speckle index value that is a statistical index value for a luminance value of a speckle;
a selection means configured to select any one of a first mode corresponding to the first parameter value and a second mode corresponding to the second parameter value;
a calculation means configured to calculate the speckle index value on a basis of the speckle image and the first parameter value in a case where the first mode is selected, and to calculate the speckle index value on a basis of the speckle image and the second parameter value in a case where the second mode is selected;
a generation means configured to generate a speckle index value image on a basis of the calculated speckle index value; and
a display control means configured to allow a display unit to display the speckle index value image.

4. The information processing device according to claim 3, wherein
the first parameter value is a parameter value corresponding to first velocity assumed as velocity of fluid in the subject, and
the second parameter value is a parameter value corresponding to second velocity lower than the first velocity assumed as velocity of fluid in the subject.

5. The information processing device according to claim 3, wherein
the acquisition means further acquires a visible light image from an imaging means that images reflected light of incoherent visible light with which the subject is irradiated, and
the display control means allows the display unit to display the speckle index value image and the visible light image in parallel or in a superimposed manner.

6. The information processing device according to claim 3, wherein the selection means selects any one of the first mode and the second mode according to an operation by a user.

7. The information processing device according to claim 3, wherein the selection means selects one of the first mode and the second mode in which a dynamic range of the speckle index value in a region of interest of the speckle image is larger.

8. The information processing device according to claim 3, wherein the selection means checks histogram distribution of the speckle index value in a region of interest of the speckle image in the first mode and the second mode, and, in a case where there are two peaks, selects one of the modes in which a centroid distance between the two peaks is larger.

9. An information processing method using a first parameter value and a second parameter value different from each other as values of a parameter for calculating a speckle index value that is a statistical index value for a luminance value of a speckle, the method comprising:
an acquisition step of acquiring a speckle image from an imaging means that images reflected light of coherent light with which a subject is irradiated;
a selection step of selecting any one of a first mode corresponding to the first parameter value and a second mode corresponding to the second parameter value;
a calculation step of calculating the speckle index value on a basis of the speckle image and the first parameter value in a case where the first mode is selected, and calculating the speckle index value on a basis of the speckle image and the second parameter value in a case where the second mode is selected;
a generation step of generating a speckle index value image on a basis of the calculated speckle index value; and
a display control step of allowing a display unit to display the speckle index value image.
